(19) United States
(12) Patent Application Publication
(10) Pub. No.: US 2024/0139478 A1
(43) Pub. Date: May 2, 2024

(54) SHAPE MEMORY ACTUATORS FOR ADJUSTABLE SHUNTING SYSTEMS, AND ASSOCIATED SYSTEMS AND METHODS

(71) Applicant: Shifamed Holdings, LLC, Campbell, CA (US)

(72) Inventors: Scott Robertson, Portland, OR (US); Brian Fahey, Menlo Park, CA (US); Bhumica Amin, San Francisco, CA (US)

(21) Appl. No.: 18/548,147

(22) PCT Filed: Mar. 9, 2022
(86) PCT No.: PCT/US2022/019526

§ 371 Date: Aug. 28, 2023

Related U.S. Application Data
(60) Provisional application No. 63/158,530, filed on Mar. 9, 2021.

(51) Int. Cl. A61M 27/00

(52) U.S. Cl. CPC A61M 27/002; A61M 2205/0266; A61M 2205/3368; A61M 2205/36

(57) ABSTRACT

The present technology is generally directed to systems and methods for transporting fluid from a first body region to a second body region, and in particular to shape memory actuators for adjustable shunting systems. The shape memory actuators can have a hysteresis temperature window that surrounds body temperature. For example, the shape memory actuator can be composed at least in part of Nitinol or a Nitinol alloy and have a low-temperature-phase finish-transformation-temperature (e.g., M_{f}) that is less than body temperature and a high-temperature-phase finish-transformation-temperature (e.g., A_{f}) that is greater than body temperature.

### CROSS-REFERENCE TO RELATED APPLICATION(S)

This application claims the benefit of U.S. Provisional Application No. 63/158,530, filed Mar. 9, 2021, the disclosure of which is incorporated by reference herein in its entirety.

### TECHNICAL FIELD

The present technology is generally directed to shape memory actuators for adjustable shunting systems.

### BACKGROUND

Implantable shunting systems are widely used to treat a variety of patient conditions by shunting fluid from a first body region/cavity to a second body region/cavity. The flow of fluid through the shunting systems is primarily controlled by the pressure gradient across the shunt lumen and the geometry (e.g., size) of the shunt lumen. One challenge with conventional shunting systems is selecting the appropriate geometry of the shunt lumen for a particular patient. A lumen that is too small may not provide enough therapy to the patient, while a lumen that is too large may create new issues in the patient. Despite this, most conventional shunts cannot be adjusted once they have been implanted. Accordingly, once the system is implanted, the therapy provided by the shunting system cannot be adjusted or titrated to meet the patient's individual needs.

As a result of the above, shunting systems with adjustable lumens have recently been proposed to provide a more personalized or titratable therapy. Such systems enable clinicians to titrate the therapy to an individual patient's needs, as well as adjust the therapy over time as the patient's disease changes. Adjustable shunting systems, however, generally require energy to drive the adjustment. Energy can be delivered invasively (e.g., energy delivered via a catheter) or non-invasively (e.g., energy delivered by an implanted battery via induction). The energy required to adjust the shunt varies depending on the actuation mechanism incorporated into the shunting system.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a graph depicting an energy profile of Nitinol at various temperatures.

FIG. 2 is a graph depicting a temperature hysteresis window of a Nitinol actuator configured in accordance with select embodiments of the present technology.

FIGS. 3A and 3B illustrate a representative implantable shunting system having a shape memory actuator and configured in accordance with select embodiments of the present technology.

FIG. 4 is a graph depicting an energy profile of Nitinol for a shape memory actuator having an M_{f} above body temperature.

FIG. 5 is a graph depicting an energy profile of Nitinol for a shape memory actuator having an M_{f} below body temperature and configured in accordance with select embodiments of the present technology.

### DETAILED DESCRIPTION

The present technology is generally directed to systems and methods for transporting fluid from a first body region to a second body region and, in particular, to shape memory actuators for adjustable shunting systems. Shape memory actuators configured in accordance with the present technology can have a hysteresis temperature window that surrounds body temperature. For example, the shape memory actuator can be composed at least in part of Nitinol or a Nitinol alloy and have a low-temperature-phase finish-transformation-temperature (e.g., a martensite finish temperature (M_{f})) that is less than body temperature and a high-temperature-phase finish-transformation-temperature (e.g., an austenite finish temperature (A_{f})) that is greater than body temperature. In some embodiments, the shape memory actuator has a M_{f} that is at least 10° C. less than body temperature and an A_{f} that is at least 10° C. above body temperature. In some embodiments, the shape memory actuator is manufactured such that the temperature differential between M_{f} and body temperature and the temperature differential between A_{f} and body temperature are ab out the same, or within about 10%, or within ab out 20%. For example, in a particular embodiment the shape memory actuator can have an M_{f} of about 10° C. and an A_{f} of about 60° C. In other embodiments, however, the M_{f} and/or the A_{f} can vary.

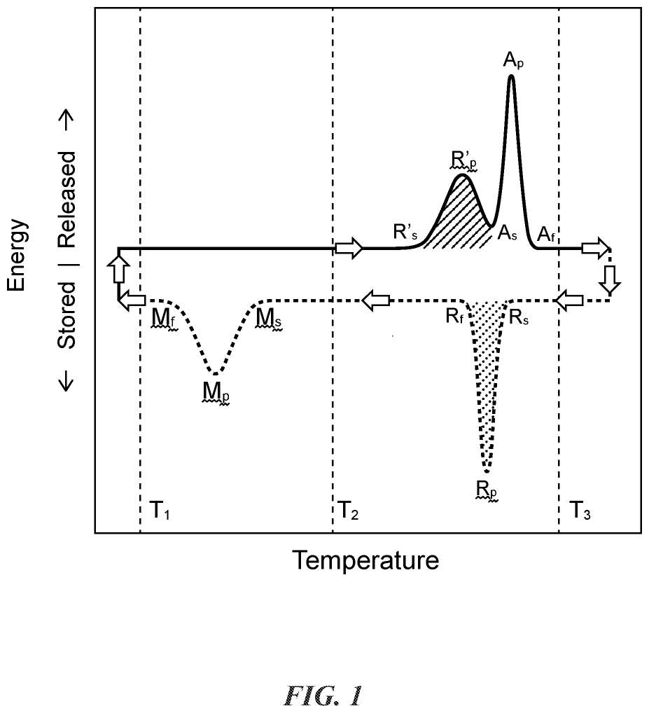

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1-5. In various respects, the terminology used to describe shape memory behavior may adopt the conventions described in ASTM F2005 (Standard Terminology for Nickel-Titanium Shape Memory Alloys). Although the terminology adopted herein is from Nickel-Titanium alloys, it is understood that the invention is not limited to Nickel-Titanium alloys (e.g., Ti—Nb, Ni—Ti—Cu, Co—Al—Ni, Ag—Cd, Au—Cd, and various polymeric shape memory materials may be substituted).

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

As used herein, the term “geometry” can include the size and/or the shape of an element. Accordingly, when the present disclosure describes a change in geometry, it can refer to a change in the size of an element (e.g., moving from a smaller circle to a larger circle), a change in the shape of an element (e.g., moving from a circle to an oval), and/or a change in the shape and size of an element (e.g., moving from a smaller circle to a larger oval). In various embodiments, “geometry” refers to the relative arrangements and/or positions of elements in the respective system.

As used herein, “malleable” and “relatively malleable” refers to physical properties of an actuator existing in one phase (e.g., a martensitic, mostly martensitic, or R-phase) and thus being generally deformable via the application of a force (e.g., balloon expansion), and “thermoelastic recovery” refers to heating the actuator such that it transforms to another phase (e.g., R-phase, austenitic, or mostly R-phase or austenitic phase) and recovers its pre-defined geometry.

The headings provided herein are for convenience only and do not interpret the scope or meaning of the claimed present technology.

### A. Nitinol Energy Profile

FIG. 1 shows a representative energy profile of Nitinol (and its alloys) as determined by Differential Scanning Calorimetry (DSC). In particular, FIG. 1 illustrates the energy profile when the material is heated from martensite (shown in solid line) and when cooled from austenite (shown in broken line). On heating from martensite, R′ₛ is the temperature at which martensite starts to transform to R-phase, R′ₚ is the peak of R-phase transformation, R′f (not shown due to overlap with the austenite peak in FIG. 1) is the finish of the R-phase transformation, Aₛ is the temperature at which R-phase (or martensite if R-phase is absent) starts to transform to austenite, Aₚ is the peak of austenite transformation, and A_{f} is the temperature at which all R-phase (or martensite if R-phase is absent) has transformed to austenite. On cooling from austenite, Rₛ is the temperature at which austenite starts to transform to R-phase, Rₚ is the peak of R-phase transformation, R_{f} is the temperature at which all austenite has transformed to R-phase, Mₛ is the temperature at which R-phase (or austenite if R-phase is absent) starts to transform to martensite, Mₚ is the peak of martensite transformation, and M_{f} is the temperature at which all R-phase (or austenite if R-phase is absent) has transformed to martensite.

To those skilled in the art, it will be recognized that although shown here for completeness, R-phase peaks (colored with cross-hatching) are not always observed in Nitinol and its alloys; e.g., in some materials austenite transforms directly to martensite, and vice-versa, upon cooling and heating, respectively. Moreover, it will be recognized that the relative positions of each peak may shift with alloy formulation and/or processing, such that the illustrated curves described herein are just one representative embodiment with others excluded for brevity. For example, A_{f} will always be greater than M_{f}. However, Aₛ may be greater than or less than Mₛ. The widths, heights, and overlap of each peak may also vary from the illustrated profile.

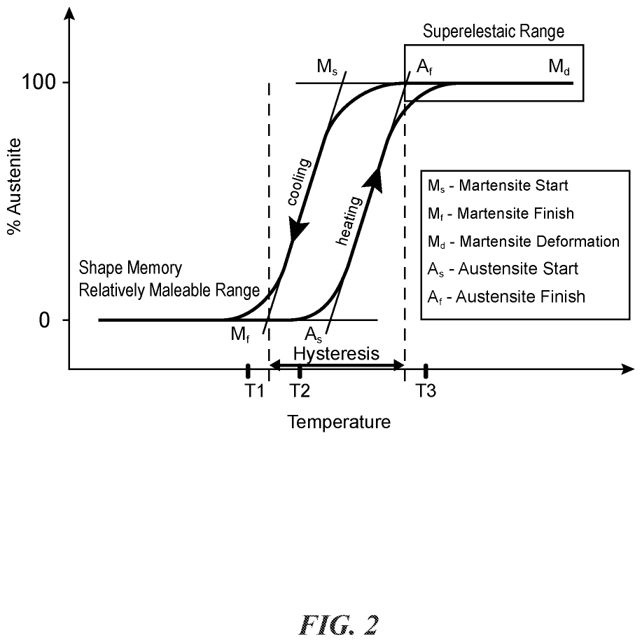

As will be described in greater detail below, the present technology utilizes the temperature path dependency of Nitinol's phases, sometimes referred to as the temperature hysteresis (e.g., see FIG. 2). Specifically, when a material begins at a high temperature (e.g., at T₃ which is above A_{f}) and is then cooled below a lower temperature (e.g., below Mₛ) it begins to transform from the high-temperature phase (austenite or R-phase) to the low-temperature phase (R-phase or martensite). When cooled below M_{f} (e.g., to T₁) the entire microstructure or substantially all of the microstructure is transformed to martensite. Once the actuator is in a lower-temperature material phase (e.g., martensite or R-phase), a temperature hysteresis, or superheating, is required to reverse the transformation from the low-temperature phase (e.g., martensite or R-phase) back to the high-temperature phase (e.g., R-phase or austenite).

As one skilled in the art will appreciate, this description is an idealized phase transformation. In reality, some small fraction of material will remain untransformed due to microstructural barriers to the transformation (e.g., residual stresses, inclusions, preferred grain boundary orientation, etc.). This incomplete transformation is acknowledged to be relevant throughout the discussion, but the idealized scenario is described herein for brevity.

### B. Shape Memory Actuators Having Optimized Energy Profiles

Shape memory actuators can be used to make in vivo adjustments to a geometry of an implanted shunting system fluidly connecting a first body region and a second body region. For example, if a shape memory actuator is deformed relative to its preferred geometry, the shape memory actuator can be thermally actuated by heating at least a portion of the actuator to induce a geometric change in the actuator (e.g., to and/or toward its preferred geometry). More specifically, shape memory actuators are actuated by heating the deformed shape memory actuator or a portion thereof above a transition temperature to induce a material phase change therein (e.g., the shape memory actuator transitions from a low-temperature martensitic or R-phase material state to a high-temperature R-phase or austenitic material state). The material phase change drives the geometry change to and/or toward the preferred geometry. The geometric change in the actuator can be translated into a geometric change in the shunt lumen and/or lumen orifice.

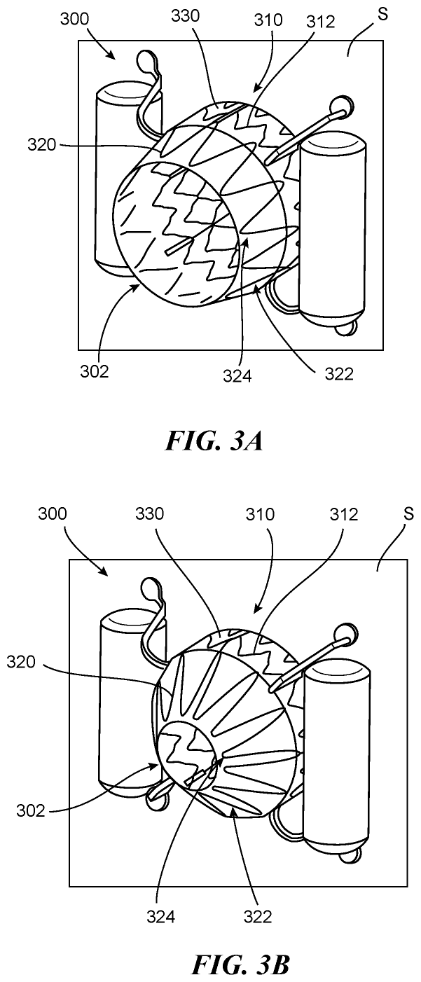

FIGS. 3A and 3B, for example, illustrate a representative implantable shunting system 300 having a shape memory actuator and configured in accordance with select embodiments of the present technology. Referring collectively to FIGS. 3A and 3B, the system 300 includes a shunt body 310, a shape memory actuator 320, and one or more membranes 330 coupled to the shunt body 310 and/or the shape memory actuator 320. The shunt body 310 can be configured to extend across a septal wall S (e.g., to fluidly connect a left atrium and a right atrium of a patient's heart) or other suitable tissue structure, and may include a frame 312 or other feature to provide structural integrity.

In some embodiments, the shape memory actuator 320 can include an actuation element or region 322 and a control element or region 324. In the illustrated embodiment, the control element 324 is the portion of the actuator 320 that at least partially defines (e.g., in combination with the membrane 330) the shape of a lumen opening 302. In the illustrated embodiment, the control element 324 is contiguous with the actuation element 322, although in other embodiments the control element 324 need not be contiguous or integral with the actuation element 322.

The actuation element 322 can be configured to undergo the geometric change when heated from below the transition temperature to above the transition temperature. For example, the actuation element 322 can transition between a first geometric configuration corresponding to a relatively larger diameter orifice (FIG. 3A) and a second geometric configuration corresponding to a relatively smaller diameter orifice (FIG. 3B). As set forth above, the control element 324 is coupled to (e.g., contiguous with, integral with) the actuation element 322 such that the control element 322 moves in a first direction (e.g., radially outward, radially inward, etc.) in response to the actuation element 322 undergoing the geometric change. In some embodiments, shape memory actuators may include a first actuation element and a second actuation element that are independently actuatable, with the first actuation configured to move the control element in a first direction and the second actuation element configured to move the control element in a second direction different than (e.g., opposite to) the first direction. Examples of shape memory actuators and adjustable shunting systems utilizing shape memory actuators are described in U.S. patent application Ser. No. 17/016,192, filed Sep. 9, 2020, and Ser. No. 17/524,631, filed Nov. 11, 2021, and International Patent Application No. PCT/US2020/063360, filed Dec. 4, 2020, the disclosures of which are incorporated by reference herein in their entireties.

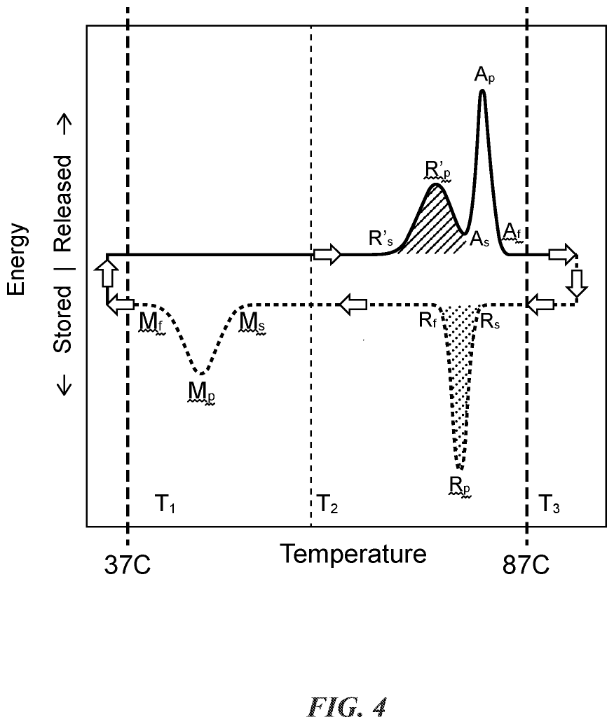

Some shape memory actuators include an M_{f} above body temperature such that the actuator is martensitic and thus relatively malleable/deformable when implanted. FIG. 4, for example, illustrates an energy profile of a Nitinol shape memory actuator manufactured to have an M_{f} above body temperature (e.g., 37° C.). To actuate the actuator, the actuator is heated above a “transition temperature,” e.g., to a temperature above R′ₛ (not shown in FIG. 4), or preferably above A_{f} (e.g., to a temperature corresponding to T₃ in FIG. 4, shown as about 87° C.). Thus, only upon significant heating would a geometry change toward the actuator's pre-defined shape or other preferred geometry be induced. One shortcoming of this method of actuation is that a relatively large temperature excursion is required to actuate the actuator (e.g., heating to 70° C. or more to reach even R′ₛ). These high temperature requirements can complicate the design of adjustment tools and the interfaces with the actuator and surrounding components. These high temperatures may also be dangerous to surrounding patient tissue, depending on the design of the shunting system and implant location.

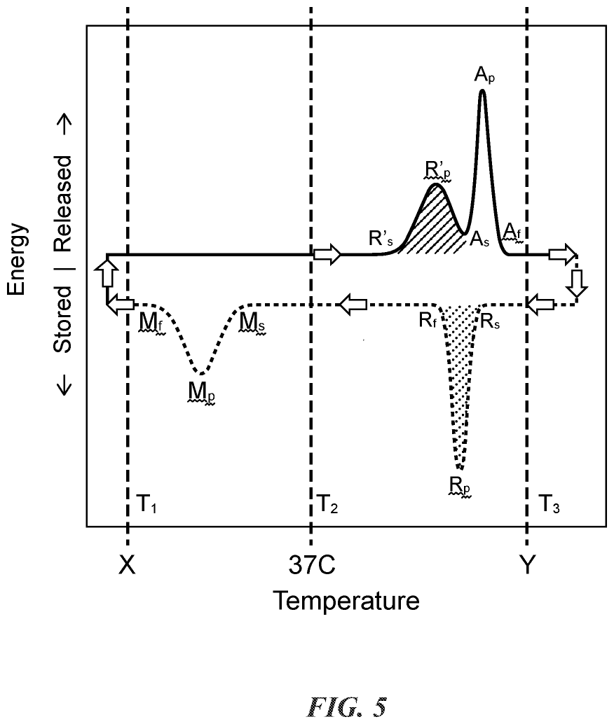

In contrast, the present technology includes shape memory actuators tuned such that body temperature is between a low-temperature-phase transformation temperature (e.g., M_{f}) and a-high-temperature phase transformation temperature (e.g., A_{f}). FIG. 5, for example, illustrates an energy profile of a Nitinol shape memory actuator manufactured to have an M_{f} of at, or below, a first temperature X that is below body temperature (e.g., at or below about 10° C.) and an A_{f} at a second temperature Y that is above body temperature (e.g., at or above about 60° C.). Because A_{f} is closer to body temperature than in the embodiment described with respect to FIG. 4, the actuator requires less heat to actuate once the system is implanted in the patient. For example, the actuator need only be heated from about 37° C. (body temperature) to about the second temperature X (e.g., about 60° C. (A_{f})) to induce a full or substantially full geometric change in the actuator. In some embodiments, the actuator can be tuned such that it only needs to be heated to about 50-60° C. to induce the full or the substantially full geometric change, with partial geometric change achieved at 40-50° C., commensurate with raising the actuator temperature above the R′ₛ temperature.

In some embodiments, the shape memory actuators are specifically tuned to have hysteresis temperature window that “surrounds” body temperature. The actuators can be tuned such that the temperature differential between M_{f} and body temperature is the same or about the same as the temperature differential between A_{f} and body temperature. For example, in some embodiments, the actuator is tuned such that M_{f} is about 12° C. and A_{f} is about 62° C., so that the temperature differential between body temperature and M_{f} and the temperature differential between body temperature and A_{f} are both ab out 25° C. Of course, the actuator can be tuned such that the hysteresis temperature window does not have body temperature at its center point, but such that body temperature is still between M_{f} and A_{f}. For example, the actuator may be tuned to have an M_{f} of less than 37° C. (e.g., below 5° C., between about 5° C. to about 35° C., between about 5° C. and about 25° C., between about 5° C. and about 15° C.) and an A_{f} of greater than 37° C. (e.g., between about 40° C. and about 80° C., between about 45° C. and about 70° C., about 45° C. and about 65° C., or between about 50° C. and about 60° C.). In some embodiments, M_{f} is at least 10° C. less than body temperature and A_{f} is at least 10° C. greater than body temperature.

The present technology therefore includes manufacturing an actuator with a specific tuning of the transformation temperatures, e.g., to have body temperature reside within a hysteresis temperature window. Tuning of the actuator to achieve this configuration can be accomplished by chemical formulation (e.g., the ratio of Nickel to Titanium, and/or the addition of alloying elements such as copper, cobalt, chromium, etc.), raw material processing parameters (e.g., ingot melt size, amount of hot and cold work, forming stresses, and annealing times and temperatures), and/or finished product processing parameters (e.g., shape setting strains, stresses, times, and temperatures). Some embodiments include Nitinol alloys in the titanium-rich condition including, but not limited to, Ni_{49.5}Ti_{50.5}, Ni_{49.6}Ti_{50.4}, Ni_{49.7}Ti_{50.3}, Ni_{49.8}Ti_{50.2}, Ni_{49.9}Ti_{50.1}, Ni₄₃Ti₅₀CU₇, Ni₄₂Ti₅₀CU₈, Ni₄₁Ti₅₀CU₉, and Ni₄₀Ti₅₀Cu₁₀. One embodiment includes an equal Nickel-to-Titanium ratio of Ni₅₀Ti₅₀. Some embodiments include Nitinol chemical formulations in the Nickel-rich conditioning traditionally producing superelastic (austenitic or R-phase) behavior at body temperature, but which have been thermo-mechanically processed to have body temperature reside within a hysteresis temperature window, including but not limited to, Ti_{49.5}Ni_{50.5}, Ti_{49.6}Ni_{50.4}, Ti_{49.7}Ni_{50.3}, Ti_{49.8}Ni_{50.2}, and Ti_{49.9}Ni_{50.1}. To promote microstructural homogeneity, ingot sizes can be kept small (e.g., 2 kg, 5 kg, 10 kg, 50 kg, 100 kg), but may be made in larger more conventional sizes for manufacturing scalability (e.g., 500-3000 kg). In some embodiments, hot forming (e.g., forging, extrusion, swaging, rolling, drawing) can be performed to significantly reduce the overall size of the ingot to near its target final dimension. Subsequent cold forming (e.g., drawing, rolling) can impart the desired cold work ranging from 10-60%.

Of note, if the actuator is tuned as described above and as represented energetically by FIG. 5, it can be a different phase at body temperature depending on the thermal pathway at which it arrived at body temperature. Specifically, if the actuator was cooled to T₁ (M_{f}) then heated to T₂ (body temperature), it would exist in the martensitic phase and be relatively malleable at T₂. Conversely, if the actuator was heated to T₃ (A_{f}) then cooled to T₂, it would exist in the austenitic- or R-phase and be generally superelastic at body temperature and/or less malleable at body temperature than the previous thermal pathway. This is in contrast to actuators having an M_{f} above body temperature, which will be martensitic at body temperature regardless of any thermal pathway at which it arrived at body temperature.

If the actuator is austenitic at body temperature, the actuator can advantageously have superelastic properties upon implantation (as opposed to being relatively malleable). This is beneficial because the actuator is generally crimped to a reduced diameter in order to install it into a catheter for delivery. If deployed into the target anatomy in the malleable state, the actuator will remain (generally) in its crimped diameter. In contrast, if the actuator is in a less malleable condition (e.g., superelastic) in the crimped configuration, then it would rebound (expand) nearer to its desired neutral position upon deployment into the target anatomy.

One such embodiment is contemplated by the following example. An actuator is manufactured to have a pre-defined 5 mm diameter. It is crimped down to 3 mm diameter to be placed into the catheter. If that actuator was chilled to T₁, then heated to body temperature, the actuator would be martensitic when deployed and would remain approximately 3 mm when deployed. Instead, if the actuator were heated to T₃ (e.g., via a warm saline flush prior to implantation), the actuator would be austenitic (or R-phase) at body temperature, and would expand nearer to its 5 mm diameter upon release from the catheter. In either case, the actuator can subsequently be cooled or heated in vivo to permit easier mechanical deformation and thermoelastic recovery of the actuator, respectively, described below.

If the thermal pathway rendered the actuator to be austenitic at body temperature (i.e., if it were first heated to T₃ prior to cooling to 37° C.), it generally must be cooled to the relatively malleable phase (e.g., to T₁) before, or during, mechanically deforming the actuator if the imparted deformations wish to remain once the mechanically deforming force is removed (e.g., through balloon expansion to increase the effective diameter of the shunt followed by dilation of the balloon). Cooling may be performed either prior to, or during, the application of mechanical force. Imparting this relatively malleable condition to the actuator may be achieved before ever implanting the device—for example, if T₁>room temperature, then simply having the implant at room temperature prior to implant will result in the relatively malleable condition. If T₁<room temperature, then the implant may be exposed to ice or any other cooling mechanism known in the field to be available during an implantation procedure. Once implanted, there are numerous methods for cooling the implant to T₁ including, but not limited to, room temperature saline flush, chilled saline flush, cooling balloon, etc. When the user wishes to return the device to its predefined shape or geometry, the actuator can be heated to raise the actuator's temperature to T₃. Heating mechanisms can include resistive heating, inductive heating, heating via warm saline, or other suitable techniques, such as those described in U.S. patent application Ser. Nos. 17/016,192 and 17/524,631, and International Patent Application No. PCT/US2020/063360, all previously incorporated by reference herein.

As one of skill in the art will appreciate from the disclosure herein, various components of the systems described above can be omitted without deviating from the scope of the present technology. Likewise, additional components not explicitly described above may be added to the systems without deviating from the scope of the present technology. Accordingly, the present technology is not limited to the configurations expressly identified herein, but rather encompasses variations and alterations of the described systems.

The technology described herein can be used in adjustable shunting systems adapted for use at a variety of anatomical locations. For example, in some embodiments the adjustable shunting systems can be interatrial shunts configured to extend through a septal wall and shunt blood from a left atrium to a right atrium. In other embodiments, the adjustable shunting systems can be positioned at another location, such as to provide blood flow between other chambers and passages of the heart or other parts of the cardiovascular system. For example, the systems described herein can be used to shunt blood between the left atrium and the coronary sinus, or between the right pulmonary vein and the superior vena cava, or from the right atrium to the left atrium. In yet other embodiments, the adjustable shunting systems may be used to shunt fluid between other body regions.

### EXAMPLES

Several aspects of the present technology are set forth in the following examples:
1. An implantable medical device for treating a human subject, the implantable medical device comprising:
   a shape memory actuator configured to adjust fluid flow through an adjustable shunting system within the human subject, wherein the shape memory actuator is tuned to have a low-temperature-phase transformation-temperature below body temperature and a high-temperature-phase transformation-temperature above body temperature, and
   wherein the low-temperature-phase transformation-temperature is at least 10° C. less than body temperature and the high-temperature-phase transformation-temperature is at least 10° C. greater than body temperature.
2. The implantable medical device of example 1 wherein the shape memory actuator includes an actuation element and a control element, and wherein:
   at least the actuation element is tuned to have the high-temperature-phase transformation-temperature above body temperature and the low-temperature-phase transformation-temperature below body temperature,
   the actuation element is configured to undergo a geometric change when heated from below the high-temperature-phase-transformation-temperature to above the high-temperature-phase-transformation temperature, and
   the control element is configured to move in a first direction in response to the actuation element undergoing the geometric change.
3. The implantable medical device of example 2 wherein the actuation element and the control element are contiguous.
4. The implantable medical device of example 3 wherein the actuation element is a first actuation element, the shape memory actuator further comprising a second actuation element, wherein:
   the second actuation element is tuned to have the high-temperature-phase transformation-temperature above body temperature and the low-temperature-phase transformation-temperature below body temperature;
   the second actuation element is configured to undergo a geometric change when heated from below the high-temperature-phase-transformation-temperature to above the high-temperature-phase-transformation temperature, and
   the control element is configured to move in a second direction in response to the second actuation element undergoing the geometric change, the second direction being different than the first direction.
5. The implantable medical device of any of examples 1-4 wherein the low-temperature-phase is martensitic and the high-temperature-phase is austenitic.
6. The implantable medical device of any of examples 1-4 wherein the low-temperature phase is martensitic and the high-temperature-phase is R-phase.
7. The implantable medical device of any of examples 1-4 wherein the low-temperature phase is R-phase and the high-temperature-phase is austenitic.
8. The implantable medical device of any of examples 1-7 wherein the high-temperature-phase transformation-temperature is between about 45° C. and about 65° C.
9. The implantable medical device of any of examples 1-8 wherein the low-temperature-phase transformation-temperature is at or below 25° C.
10. The implantable medical device of any of examples 1-9 wherein the low-temperature-phase transformation-temperature is above about 5° C.
11. The implantable medical device of any of examples 1-10 wherein the shape memory actuator is configured such that a first temperature differential between the low-temperature-phase transformation-temperature and body-temperature is about the same as a second temperature differential between the high-temperature-phase transformation-temperature and body-temperature.
12. The implantable medical device of any of examples 1-11 wherein the shape memory actuator is capable of being at least two differing phases at body temperature, and wherein:
   the shape memory actuator is in its high-temperature-phase at body temperature if the shape memory actuator was heated above a high-temperature-phase finish-transformation-temperature before being cooled to body temperature, wherein body temperature is above a starting-transformation-temperature of the low-temperature-phase; and
   the shape memory actuator is in its low-temperature-phase at body temperature if the shape memory actuator was cooled below a low-temperature-phase finish-transformation-temperature before being heated to body temperature, wherein body temperature is below a starting-transformation-temperature of the high-temperature-phase.
13. The implantable medical device of any of examples 1-12 wherein the shape memory actuator comprises Nitinol.
14. The implantable medical device of any of examples 1-12 wherein the shape memory actuator comprises a Nitinol alloy.
15. The implantable medical device of any of examples 1-14 wherein the shape memory actuator is coupled to the adjustable shunting system and, during operation, is configured to change a geometry of a lumen extending through the adjustable shunting system.
16. An implantable shape memory actuator for treating a human subject, wherein the implantable shape memory actuator is configured to adjust fluid flow through an adjustable shunting system, and wherein the shape memory actuator is tuned to have:
   a martensite finish temperature (M_{f}) at least 10° C. less than body temperature,
   a R-phase finish temperature (R_{f}) above body temperature, and
   an austenite finish temperature (A_{f}) above body temperature.
17. The shape memory actuator of example 16 wherein the A_{f} is between about 45° C. and about 65° C.
18. The shape memory actuator of example 16 or example 17 wherein the M_{f} is at or below 25° C.
19. The shape memory actuator of any of examples 16-18 wherein the M_{f} is above about 5° C.
20. A method of adjusting fluid flow through a shunting system implanted between a first body region and a second body region, the method comprising:
   cooling a shape memory actuator of the shunting system in vivo; and
   mechanically deforming the shape memory actuator to change fluid flow through the shunting system.
21. The method of example 20 wherein cooling the shape memory actuator includes cooling the shape memory actuator below a low-temperature-phase finish-transformation-temperature of the shape memory actuator.
22. The method of example 21 wherein the low-temperature-phase finish-transformation-temperature is less than or equal to 25° C.
23. The method of example 21 or example 22 wherein the low-temperature-phase finish-transformation-temperature is above about 5° C.
24. The method of any of examples 20-23 wherein cooling the shape memory actuator includes:
   advancing a cooling tool to the shape memory actuator; and
   cooling the shape memory actuator using the cooling tool.
25. The method of any of examples 20-24 wherein mechanically deforming the shape memory actuator includes mechanically expanding the shape memory actuator.
26. The method of any of examples 20-25 wherein mechanically deforming the shape memory actuator includes deforming the shape memory actuator to a first geometry, the method further comprising:
   heating the shape memory actuator of the shunting system in vivo to cause the shape memory actuator to assume a second geometry different than the first geometry.
27. The method of example 26 wherein heating the shape memory actuator includes heating the shape memory actuator above a high-temperature-phase finish-transformation-temperature of the shape memory actuator.
28. The method of example 27 wherein the high-temperature-phase finish-transformation-temperature is between about 45° C. and about 60° C.
29. A method of implanting a shape memory implant into a patient, the method comprising:
   crimping the shape memory implant and positioning the shape memory implant in a catheter; and
   after positioning the shape memory implant in the catheter and before inserting the catheter into the patient, heating the shape memory implant to a temperature greater than body temperature.
30. The method of example 29 wherein heating the shape memory implant includes heating the shape memory implant above a high-temperature-phase finish-transformation-temperature.
31. The method of example 30, further comprising implanting the shape memory implant into the patient, wherein the shape memory implant is relatively non-malleable at body temperature by virtue of heating the shape memory implant to the temperature greater than body temperature before inserting the catheter into the patient.
32. The method of example 31 wherein the shape memory implant is superelastic at body temperature by virtue of heating the shape memory implant to the temperature greater than body temperature.
33. A method of confirming a material state of a shape memory implant in a patient, comprising:
   cooling the shape memory actuator to transition the shape memory actuator to a low-temperature-phase; and
   confirming the shape memory actuator is in the low-temperature-phase by—
   expanding a balloon positioned within the shape memory actuator to deform the shape memory actuator, and
      deflating the balloon, wherein if the shape memory actuator exhibits little to no spring-back or recoil, the shape memory actuator is in the low-temperature-phase.
34. The method of example 20 wherein the cooling step and the confirming step are performed using the same tool.

### Conclusion

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, Wi-Fi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

Unless the context clearly requires otherwise, throughout the description and the examples, the words “comprise,” “comprising,” and the like are to be construed in an inclusive sense, as opposed to an exclusive or exhaustive sense; that is to say, in the sense of “including but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. An implantable medical device for treating a human subject, the implantable medical device comprising:
a shape memory actuator configured to adjust fluid flow through an adjustable shunting system within the human subject, wherein the shape memory actuator is tuned to have a low-temperature-phase transformation-temperature below body temperature and a high-temperature-phase transformation-temperature above body temperature, and
wherein the low-temperature-phase transformation-temperature is at least 10° C. less than body temperature and the high-temperature-phase transformation-temperature is at least 10° C. greater than body temperature.

2. The implantable medical device of claim 1 wherein the shape memory actuator includes an actuation element and a control element, and wherein:
at least the actuation element is tuned to have the high-temperature-phase transformation-temperature above body temperature and the low-temperature-phase transformation-temperature below body temperature,
the actuation element is configured to undergo a geometric change when heated from below the high-temperature-phase-transformation-temperature to above the high-temperature-phase-transformation temperature, and
the control element is configured to move in a first direction in response to the actuation element undergoing the geometric change.

3. The implantable medical device of claim 2 wherein the actuation element and the control element are contiguous.

4. The implantable medical device of claim 3 wherein the actuation element is a first actuation element, the shape memory actuator further comprising a second actuation element, wherein:
the second actuation element is tuned to have the high-temperature-phase transformation-temperature above body temperature and the low-temperature-phase transformation-temperature below body temperature;
the second actuation element is configured to undergo a geometric change when heated from below the high-temperature-phase-transformation-temperature to above the high-temperature-phase-transformation temperature, and
the control element is configured to move in a second direction in response to the second actuation element undergoing the geometric change, the second direction being different than the first direction.

5. The implantable medical device of claim 1 wherein the low-temperature-phase is martensitic and the high-temperature-phase is austenitic.

6. The implantable medical device of claim 1 wherein the low-temperature phase is martensitic and the high-temperature-phase is R-phase.

7. The implantable medical device of claim 1 wherein the low-temperature phase is R-phase and the high-temperature-phase is austenitic.

8. The implantable medical device of claim 1 wherein the high-temperature-phase transformation-temperature is between about 45° C. and about 65° C.

9. The implantable medical device of claim 1 wherein the low-temperature-phase transformation-temperature is at or below 25° C.

10. The implantable medical device of claim 1 wherein the low-temperature-phase transformation-temperature is above about 5° C.

11. The implantable medical device of claim 1 wherein the shape memory actuator is configured such that a first temperature differential between the low-temperature-phase transformation-temperature and body-temperature is about the same as a second temperature differential between the high-temperature-phase transformation-temperature and body-temperature.

12. The implantable medical device of claim 1 wherein the shape memory actuator is capable of being at least two differing phases at body temperature, and wherein:
the shape memory actuator is in its high-temperature-phase at body temperature if the shape memory actuator was heated above a high-temperature-phase finish-transformation-temperature before being cooled to body temperature, wherein body temperature is above a starting-transformation-temperature of the low-temperature-phase; and
the shape memory actuator is in its low-temperature-phase at body temperature if the shape memory actuator was cooled below a low-temperature-phase finish-transformation-temperature before being heated to body temperature, wherein body temperature is below a starting-transformation-temperature of the high-temperature-phase.

13. The implantable medical device of claim 1 wherein the shape memory actuator comprises Nitinol.

14. The implantable medical device of claim 1 wherein the shape memory actuator comprises a Nitinol alloy.

15. The implantable medical device of claim 1 wherein the shape memory actuator is coupled to the adjustable shunting system and, during operation, is configured to change a geometry of a lumen extending through the adjustable shunting system.

16. An implantable shape memory actuator for treating a human subject, wherein the implantable shape memory actuator is configured to adjust fluid flow through an adjustable shunting system, and wherein the shape memory actuator is tuned to have:
a martensite finish temperature (M_{f}) at least 10° C. less than body temperature,
a R-phase finish temperature (R_{f}) above body temperature, and
an austenite finish temperature (A_{f}) above body temperature.

17. The shape memory actuator of claim 16 wherein the A_{f} is between about 45° C. and about 65° C.

18. The shape memory actuator of claim 16 wherein the M_{f} is at or below 25° C.

19. The shape memory actuator of claim 16 wherein the M_{f} is above about 5° C.

20. A method of adjusting fluid flow through a shunting system implanted between a first body region and a second body region, the method comprising:
cooling a shape memory actuator of the shunting system in vivo; and
mechanically deforming the shape memory actuator to change fluid flow through the shunting system.

21. The method of claim 20 wherein cooling the shape memory actuator includes cooling the shape memory actuator below a low-temperature-phase finish-transformation-temperature of the shape memory actuator.

22. The method of claim 21 wherein the low-temperature-phase finish-transformation-temperature is less than or equal to 25° C.

23. The method of claim 21 wherein the low-temperature-phase finish-transformation-temperature is above about 5° C.

24. The method of claim 20 wherein cooling the shape memory actuator includes:
advancing a cooling tool to the shape memory actuator; and
cooling the shape memory actuator using the cooling tool.

25. The method of claim 20 wherein mechanically deforming the shape memory actuator includes mechanically expanding the shape memory actuator.

26. The method of claim 20 wherein mechanically deforming the shape memory actuator includes deforming the shape memory actuator to a first geometry, the method further comprising:
heating the shape memory actuator of the shunting system in vivo to cause the shape memory actuator to assume a second geometry different than the first geometry.

27. The method of claim 26 wherein heating the shape memory actuator includes heating the shape memory actuator above a high-temperature-phase finish-transformation-temperature of the shape memory actuator.

28. The method of claim 27 wherein the high-temperature-phase finish-transformation-temperature is between about 45° C. and about 60° C.

29. A method of implanting a shape memory implant into a patient, the method comprising:
crimping the shape memory implant and positioning the shape memory implant in a catheter; and
after positioning the shape memory implant in the catheter and before inserting the catheter into the patient, heating the shape memory implant to a temperature greater than body temperature.

30. The method of claim 29 wherein heating the shape memory implant includes heating the shape memory implant above a high-temperature-phase finish-transformation-temperature.

31. The method of claim 30, further comprising implanting the shape memory implant into the patient, wherein the shape memory implant is relatively non-malleable at body temperature by virtue of heating the shape memory implant to the temperature greater than body temperature before inserting the catheter into the patient.

32. The method of claim 31 wherein the shape memory implant is superelastic at body temperature by virtue of heating the shape memory implant to the temperature greater than body temperature.

33. A method of confirming a material state of a shape memory implant in a patient, comprising:
cooling the shape memory actuator to transition the shape memory actuator to a low-temperature-phase; and
confirming the shape memory actuator is in the low-temperature-phase by—
expanding a balloon positioned within the shape memory actuator to deform the shape memory actuator, and
deflating the balloon, wherein if the shape memory actuator exhibits little to no spring-back or recoil, the shape memory actuator is in the low-temperature-phase.

34. The method of claim 20 wherein the cooling step and the confirming step are performed using the same tool.
